(19) United States
(12) Patent Application Publication
(10) Pub. No.: US 2006/0030896 A1
(43) Pub. Date: Feb. 9, 2006

(54) Pulsed electromagnetic field method of treatment of degenerative disc disease

(76) Inventors: Bruce J. Simon, Mountain Lakes, NJ (US); Jean C. Gan, Morris Township, NJ (US); James R. Pastena, Succasunna, NJ (US)

Correspondence Address: BAHRET & ASSOCIATES, 320 NORTH MERIDIAN STREET, SUITE 510, INDIANAPOLIS, IN, 46204, US

(21) Appl. No.: 11/176,589

(22) Filed: Jul. 7, 2005

Related U.S. Application Data
(60) Provisional application No. 60/586,744, filed on Jul. 9, 2004.

(51) Int. Cl. A61N 1/34; A61N 1/18

(52) U.S. Cl. 607/43; 607/72; 607/74

(57) ABSTRACT

A pulsed electromagnetic field method of treating degenerative disc disease, wherein, in one embodiment, a patient in need of treatment for degenerative disc disease is administered a pulsed electromagnetic field (PEMF) having repetitive pulse bursts approximately 5 ms in duration, with a pulse burst repetition rate of approximately 15 Hz.

### CROSS-REFERENCE TO RELATED APPLICATION

This application claims the benefit of Provisional Patent Application No. 60/586,744, filed Jul. 9, 2004, which application is hereby incorporated by reference.

### BACKGROUND OF THE INVENTION

This invention relates to methods for treating degenerative disc disease, and more particularly to methods for treating degenerative disc disease noninvasively.

Degenerative disc disease (DDD), an irreversible process, is the most common cause of back pain. The intervertebral disc consists of a gelatinous nucleus pulposus encapsulated by a fibrous annulus fibrosus and the end-plates. The nucleus pulposus plays an important role in weight transmission. When this gel-like fluid is subjected to load, the nucleus pulposus is pressurized deforming to establish an equilibrating pressure between the annulus fibrosus and vertebral end-plates. However, with maturation, the tissue loses its gel-like properties becoming less hydrated and ultimately more fibrous. It is thus less able to transmit the load by exerting pressure on the annulus fibrosus. As a result, the annulus fibrosus undergoes greater deformation, and a greater share of the vertical load is borne directly by the annulus fibrosus. These changes result in the development of cracks and cavities within the annulus fibrosus leading to degeneration.

There are 3 therapeutic approaches to date:

1) Anti-neuralgic and anti-inflammatory;

2) Minimally invasive percutaneous treatments such as discectomy, intradiscal electrothermy, nucleoplasty and percutaneous radiofrequency application; and

3) Major surgeries such as fusions, laminectomy and nucleus/disc replacements.

Anti-neuralgic and anti-inflammatory methods treat the symptoms but do not eliminate the problem. Minimally invasive percutaneous treatments such as those identified above temporarily improve the symptoms, but they do not prevent the progression of DDD and may even accelerate it in the long term. Major surgeries such as those identified above eliminate structures, risk nerve injury during the operation, also risk morbidity, and may result in accelerated disc degeneration at adjacent segments in the long term.

### SUMMARY OF THE INVENTION

The present invention uses electromagnetic field stimulation to regenerate the intervertebral disc and thus restore the mechanical properties of the spine or to reduce pain resulting from disc degeneration such as discogenic pain.

According to one aspect of the present invention, a patient diagnosed as having degenerative disc disease is administered a pulsed electromagnetic field (PEMF) with particular characteristics including repetitive pulse bursts less than approximately 30 ms in duration, and a pulse burst repetition rate greater than approximately 5 Hz, the pulse bursts generated with a drive signal including pulses each having a first-polarity portion with a pulse width less than 1 ms and a second-polarity portion with a shorter pulse width.

PEMF treatment according to the present invention may be used as an adjunct to other treatments such as implantation of cells (e.g., stem cells from various sources and intervertebral disc cells), biological factors (e.g., growth factors, peptides), scaffolds or materials (e.g., collagen, polymers, ceramics) and gene therapy.

Among other advantages of the present invention, it is a noninvasive and simple approach to relieving pain associated with the disc. It is simple to use, surgery is not required, the treatment can be administered early to prevent further progression of DDD, and it may regenerate the disc. Regeneration of the disc restores the mechanical properties of the spine and thus will not accelerate disc degeneration at adjacent segments in the long term.

These and other objects and advantages of the present invention will be more apparent upon reading the following detailed description in conjunction with the accompanying drawings.

### BRIEF DESCRIPTION OF THE DRAWINGS

FIG. 1 is a rear view of a human body with a PEMF treatment coil placed against the back of the body for treatment of degenerative disc disease according to one embodiment of the present invention.

FIG. 2 illustrates a PEMF signal for use in treatment of degenerative disc disease according to one embodiment of the present invention. FIGS. 2A and 2B illustrate an electrical drive signal and FIG. 2C illustrates a corresponding magnetic field waveform.

### DESCRIPTION OF PREFERRED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device and such further applications of the principles of the invention as illustrated therein being contemplated as would normally occur to one skilled in the art to which the invention relates.

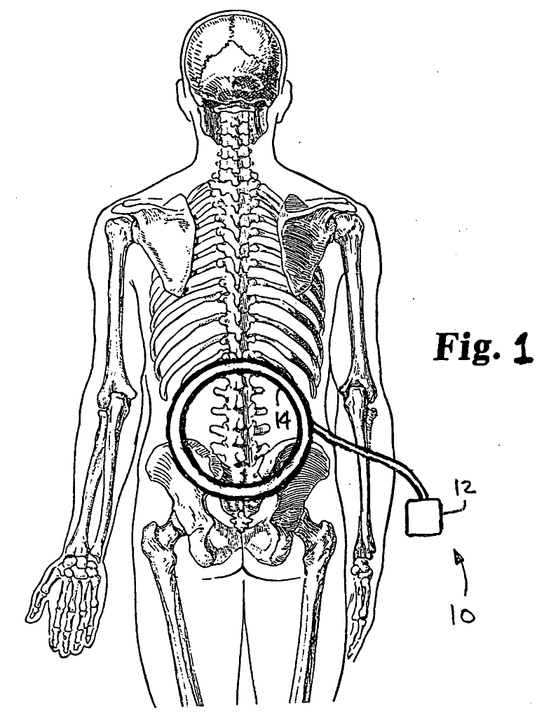

The present invention treats DDD with pulsed electromagnetic field (PEMF) stimulation. Referring to FIG. 1, one embodiment of a device 10 according to the present invention comprises a signal generator 12 which can be worn on the waist and connected to a coil 14 placed at the site of pain or location of the disc or discs to be treated. The coil may be of various sizes and configurations depending on the area to be treated. For example, a coil in the FLX family line of coils from EBI, L.P., may be placed so as to encircle a disc or discs to be treated. The coil may be held in place with a bandage or tape, for example, or with a band around the body.

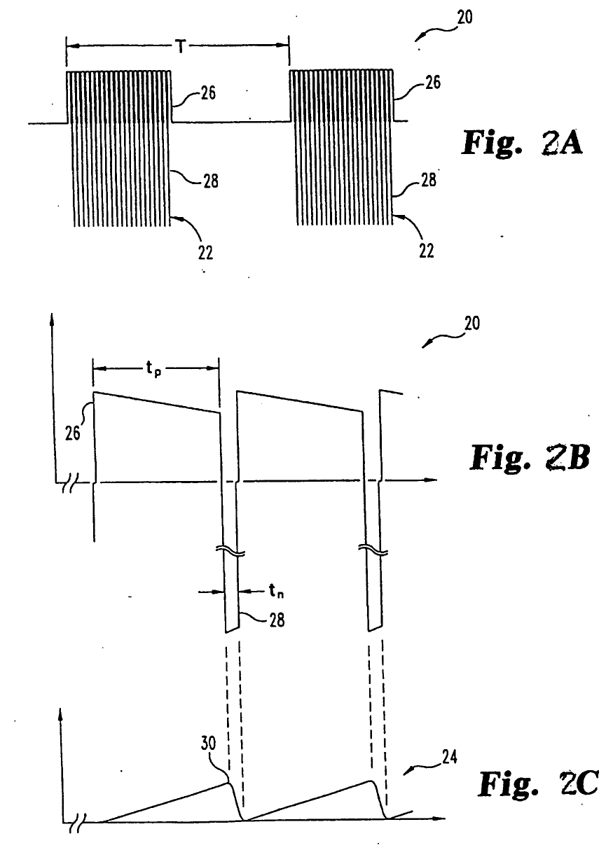

In one embodiment of the invention, the coil is supplied with an electrical drive signal such as signal 20 shown in FIGS. 2A and 2B. The signal comprises pulse bursts 22 less than approximately 30 milliseconds (ms) in duration, repeated at a rate greater than approximately 5 pulse bursts per second, i.e., a burst repetition rate greater than approximately 5 Hz. It will be appreciated that the resulting magnetic field 24 shown in FIG. 2C has these same characteristics. The drive signal has a positive portion 26 which is longer in duration than the negative portion 28. For example, the pulse width of the positive portion (tₚ) is less than 1 ms, and advantageously in the range of 100-300 microseconds (μs), whereas the pulse width of the negative portion (tₙ) is preferably less than 100 μs. A more preferred range for the positive pulse width is 200-250 μs, and the negative pulse width is more preferably in the range of 5-40 μs. The pulse bursts preferably have a duration of 2.5-5 ms and a repetition rate of 5-20 Hz.

One suitable example pulse train signal has positive and negative pulse widths of 200 μs and 24 μs, respectively, with total spacing between positive portions of 28 μs, and has 4.5-5 ms bursts repeated at 15 Hz. The peak magnetic flux density (B) 30 is preferably in the range of 0.9-1.8 milliTesla (mT) (9-18 Gauss), and more preferably approximately 1.5 mT, which is reached during the positive portion of the signal as shown in the drawing. The magnetic field is substantially unipolar in that it has little or no negative portion. Such a signal may be generated with, for example, an EBI Model 2001 Bone Healing System signal generator coupled to an EBI FLX® 1-2 or 2-2 flexible treatment coil. It will be understood that “positive” and “negative” are relative terms. A PEMF signal as described above is preferably applied for 2-12 hours per day, and more preferably approximately 8 hours per day as adjunctive therapy at least until the pain subsides and preferably until disc generation is noted in a MRI scan or the like.

Another PEMF signal with repetitive pulse bursts that may be useful in certain applications of the present invention has a pulse burst repetition rate of approximately 1.5 Hz. Examples of this signal are described in the following patent and patent application, which are both assigned to the assignee of the present invention and which are hereby incorporated by reference along with all references cited therein: U.S. Pat. No. 5,338,286 to Abbott et al., and patent application Ser. No. 10/304,721, entitled Pulsed Electromagnetic Field Stimulation Method And Apparatus With Improved Dosing, filed Nov. 26, 2002 in the name of Bruce J. Simon.

A repetitive single pulse PEMF signal with a pulse rate of approximately 72 Hz is also contemplated as useful in certain applications of the present invention. An example of this signal is described in patent application Ser. No. 10/945,291, entitled Pulsed Electromagnetic Field Method Of Treating Soft Tissue Wounds, filed Sep. 20, 2004 in the name of Bruce J. Simon and assigned to the assignee of the present invention. Other signals which may be suitable for particular applications are described in U.S. Pat. Nos. 4,105,017, 4,266,533 and 4,315,503 to Ryaby et al., which patents are hereby incorporated by reference.

While the invention is not limited to any one theory of operation, it is believed that the invention promotes disc tissue regeneration and/or inhibits further progression of DDD by, for example, inhibiting the production of inflammatory cytokines such as IL-1, or stimulating production of their antagonists such as TGF-β. As indicated above, the invention can be used as an adjunct to treatments such as implantation of cells, biological factors, scaffolds or materials, and gene therapy. The following publications are also hereby incorporated by reference along with all references cited therein: Liu, Hongxiang et al., “Pulsed Electromagnetic Fields Influence Hyaline Cartilage Extracellular Matrix Composition Without Affecting Molecular Structure,” Osteoarthritis and Cartilage, 4:63-76 (1996); Fredericks, Douglas C., et al., “Effects of Pulsed Electromagnetic Fields on Bone Healing in a Rabbit Tibial Osteotomy Model,” Journal of Orthopaedic Trauma, Vol. 14, No. 2, pp. 93-100 (2000); Ciombor, D. et al., “Modification of Osteoarthritis by Pulsed Electromagnetic Field—a Morphological Study,” OsteoArthritis and Cartilage, 11:455-462 (2003).

While the invention has been illustrated and described in detail in the drawings and foregoing description, the same is to be considered as illustrative and not restrictive in character, it being understood that only preferred embodiments have been shown and described and that all changes and modifications that come within the spirit of the invention are desired to be protected.

We claim:

1. A pulsed electromagnetic field method of treating degenerative disc disease, comprising administering to a patient in need of such treatment, with the intent to treat degenerative disc disease, a pulsed electromagnetic field having repetitive pulse bursts less than approximately 30 ms in duration, with a pulse burst repetition rate greater than approximately 5 Hz, said pulse bursts generated with a drive signal including pulses each having a first-polarity portion with a pulse width less than 1 ms and a second-polarity portion with a shorter pulse width.

2. The method of claim 1, wherein said pulse width of said first-polarity portion is in the range of approximately 100-300 μs.

3. The method of claim 2, wherein said pulse burst repetition rate is in the range of approximately 5-20 Hz.

4. The method of claim 3, wherein said pulse burst duration is in the range of approximately 2.5-5 ms.

5. The method of claim 2, wherein said pulse width of said first-polarity portion is in the range of approximately 200-250 μs, and said pulse width of said second-polarity portion is approximately 25 μs.

6. The method of claim 5, wherein said pulse burst repetition rate is approximately 15 Hz.

7. A pulsed electromagnetic field method of treating degenerative disc disease, comprising:
identifying a disc in a state of degenerative disc disease in a subject;
indicating the use of a pulsed electromagnetic field for treatment of the identified disc, the indicated electromagnetic field characterized by repetitive pulse bursts less than approximately 30 ms in duration, with a pulse burst repetition rate greater than approximately 5 Hz, said pulse bursts generated with a drive signal including pulses each having a first-polarity portion with a pulse width less than 1 ms and a second-polarity portion with a shorter pulse width.

8. The method of claim 7, wherein said pulse width of said first-polarity portion is in the range of approximately 100-300 μs.

9. The method of claim 8, wherein said pulse burst repetition rate is in the range of approximately 5-20 Hz.

10. The method of claim 9, wherein said pulse burst duration is in the range of approximately 2.5-5 ms.

11. The method of claim 8, wherein said pulse width of said first-polarity portion is in the range of approximately 200-250 μs, and said pulse width of said second-polarity portion is approximately 25 μs.

12. The method of claim 11, wherein said pulse burst repetition rate is approximately 15 Hz.

13. A pulsed electromagnetic field method of treating degenerative disc disease, comprising:
placing a treatment coil on the body of a subject at the site of an identified disc in a state of degenerative disc disease; and
applying a pulsed electromagnetic field to said disc via said treatment coil with the intent to treat the degenerative disc disease, said electromagnetic field characterized by repetitive pulse bursts less than approximately 30 ms in duration, with a pulse burst repetition rate greater than approximately 5 Hz, said pulse bursts generated with a drive signal including pulses each having a first-polarity portion with a pulse width less than 1 ms and a second-polarity portion with a shorter pulse width.

14. The method of claim 13, wherein said pulse width of said first-polarity portion is in the range of approximately 100-300 μs.

15. The method of claim 14, wherein said pulse burst repetition rate is in the range of approximately 5-20 Hz.

16. The method of claim 15, wherein said pulse burst duration is in the range of approximately 2.5-5 ms.

17. The method of claim 14, wherein said pulse width of said first-polarity portion is in the range of approximately 200-250 μs, and said pulse width of said second-polarity portion is approximately 25 μs.

18. The method of claim 17, wherein said pulse burst repetition rate is approximately 15 Hz.
